(19) United States
(12) Patent Application Publication
(10) Pub. No.: US 2017/0205427 A1
(43) Pub. Date: Jul. 20, 2017

(54) BIOMARKERS FOR ANDERSON-FABRY DISEASE

(71) Applicants: Michael West, Halifax (CA); University of British Columbia, Vancouver (CA); The Governors of The University of Alberta, Edmonton (CA)

(72) Inventors: Michael L. West, Halifax (CA); Gavin Oudit, Edmonton (CA); Bruce M. McManus, Tsawwassen (CA); Zsuzsanna Hollander, Vancouver (CA)

(21) Appl. No.: 15/328,461

(22) PCT Filed: Jul. 22, 2015
(86) PCT No.: PCT/IB2015/001804

§ 371 Date: Jan. 23, 2017

Related U.S. Application Data
(60) Provisional application No. 62/028,225, filed on Jul. 23, 2014.

(51) Int. Cl. G01N 33/68; G06F 19/00

(52) U.S. Cl. CPC G01N 33/6893; G06F 19/345; G01N 2800/245; G01N 2800/04; G01N 2800/52; G01N 2800/60

(57) ABSTRACT

Disclosed herein is a method for screening and diagnosis of Anderson-Fabry Disease in a subject based on biomarker expression in patient samples. Also disclosed are computer systems, kits, and software for implementation of the biomarkers.

### CROSS-REFERENCE

This application claims the benefit of U.S. Provisional Application No. 62/028,225, filed Jul. 23, 2014, entitled “BIOMARKERS FOR ANDERSON-FABRY DISEASE,” the entire disclosure of which is hereby incorporated herein by reference for all purposes.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file 97513_951211.TXT, created on Jul. 22, 2015, 2,641 bytes, machine format IBM-PC, MS-Windows operating system, is hereby incorporated by reference in its entirety for all purposes.

### BACKGROUND

Anderson-Fabry disease (AFD) is an X-linked lysosomal storage disorder caused by mutations in the GLA gene encoding the enzyme α-galactosidase A (α-GalA).¹ Deficiencies in α-GalA activity cause globotriaosylceramide (Gb3) to accumulate, and lead to progressive multisystem disease. Historical estimates of AFD prevalence were very low, but these have recently been recognized as underestimates in the context of multiple large-scale metabolic and genetic screening studies in Asia and Europe, wherein a high prevalence of mutations associated with late-onset or variant AFD phenotypes have been observed.²⁻⁵ Clinical manifestations of AFD may be non-specific, and, due to its rarity, other conditions are initially suspected over AFD, such that a correct diagnosis may be delayed until after irreversible end-organ damage has occurred.¹ Anderson-Fabry cardiomyopathy is the most common cause of death in AFD patients, followed by renal complications, which together highlight the need for improved diagnosis and treatment.⁶

Biomarker identification represents an expanding activity in AFD research that have the promise of addressing the present limitations to effective care that exist in delayed diagnoses.⁷ In addition to increasing diagnostic efficiency, biomarkers may offer prognostic information, or act as surrogates to monitor the effectiveness of a given treatment.^{8, 9} Whole blood, plasma and serum samples from peripheral veins offer a minimally-invasive output that reflects changes in various end-organs. In concert with techniques capable of capturing low abundance molecules, such as mass spectrometry, diagnostic algorithms may be substantially improved. Typically, the diagnosis of AFD is made based on α-GalA activity levels in peripheral blood or plasma; however, this method is unreliable in the case of variant or late-onset cases, and frequently misses the AFD diagnosis in females.¹⁰ In order to account for this, females with suspected AFD must be genetically tested to confirm the presence of a mutation associated with AFD.^{10, 11} Multiple lines of evidence, however, show that genetic testing is itself hindered by ambiguities, which further underscores the need for reliable, gender-specific biomarkers to enhance the current diagnostic algorithm.¹²

The methods and compositions of the present invention help to satisfy these and other needs for such tests.

### SUMMARY

Disclosed herein are compositions and methods for determining Anderson-Fabry Disease in a subject using biomarkers from a sample derived from the subject.

In a first aspect, disclosed herein is a method for diagnosing Anderson-Fabry Disease (AFD) in a male subject, comprising: obtaining a dataset associated with a sample obtained from the male subject, wherein the dataset comprises at least one marker selected from Table 2; analyzing the dataset to determine data for the markers, wherein the data is positively correlated or negatively correlated with a diagnosis of Anderson-Fabry Disease in the male subject.

In an embodiment, the dataset comprises data for at least two, three, four, five, six, seven, or eight markers. In another embodiment, the method further comprises determining the diagnosis of Anderson-Fabry Disease in the subject according to the relative number of positively correlated and negatively correlated marker expression level data present in the dataset.

In a second aspect, disclosed herein is a method for diagnosing Anderson-Fabry Disease (AFD) in a female subject, comprising: obtaining a dataset associated with a sample obtained from the female subject, wherein the dataset comprises at least one marker selected from Table 4; analyzing the dataset to determine data for the markers, wherein the data is positively correlated or negatively correlated with a diagnosis of Anderson-Fabry Disease in the female subject.

In an embodiment, the dataset comprises data for at least two, three, four, five, six, seven, eight or nine markers. In another embodiment, the method further comprises determining the diagnosis of Anderson-Fabry Disease in the subject according to the relative number of positively correlated and negatively correlated marker expression level data present in the dataset.

In various embodiments of the above aspects, the sample obtained from the subject is a blood sample. In various embodiments of the above aspects, the data is protein expression data. In various embodiments of the above aspects, the protein expression data is obtained using mass spectrometry or other methods

In various embodiments of the above aspects, the method is implemented using one or more computers. In various embodiments of the above aspects, the dataset is obtained stored on a storage memory.

In various embodiments of the above aspects, obtaining the dataset comprises receiving the dataset directly or indirectly from a third party that has processed the sample to experimentally determine the dataset.

In various embodiments of the above aspects, the subject is a human subject.

In various embodiments of the above aspects, the method further comprises assessing a clinical variable; and combining the assessment with the analysis of the dataset to diagnose Anderson-Fabry Disease (AFD) in the subject.

In a third aspect, disclosed herein is a method for predicting the likelihood of Anderson-Fabry Disease in a subject, comprising: obtaining a sample from a male subject, wherein the sample comprises at least one marker selected from Table 2, or obtaining a sample from a female subject, wherein the sample comprises at least one marker selected from Table 4; measuring proteins in the sample, wherein the dataset comprises protein abundance data for the markers; and analyzing the protein level data for the markers, wherein the abundance of the markers is positively correlated or negatively correlated with a diagnosis of Anderson-Fabry Disease in the subject.

In a fourth aspect, disclosed herein is a computer-implemented method for diagnosing Anderson-Fabry Disease in a subject, comprising: storing, in a storage memory, a dataset associated with a sample obtained from a male subject, wherein the dataset comprises data for at least one marker selected from Table 2, or storing, in a storage memory, a dataset associated with a sample obtained from a female subject, wherein the dataset comprises data for at least one marker selected from Table 4; and analyzing, by a computer processor, the dataset to determine the abundance of the markers, wherein the protein abundance is positively correlated or negatively correlated with a diagnosis of Anderson-Fabry Disease in the subject.

In a fifth aspect, disclosed herein is a system for diagnosing Anderson-Fabry Disease in a subject, the system comprising: a storage memory for storing a dataset associated with a sample obtained from a male subject, wherein the dataset comprises data for at least one marker selected from Table 2, or a storage memory for storing a dataset associated with a sample obtained from a female subject, wherein the dataset comprises data for at least one marker selected from Table 4; and a processor communicatively coupled to the storage memory for analyzing the dataset to determine the abundance of the markers, wherein the protein abundance are positively correlated or negatively correlated with a diagnosis of Anderson-Fabry Disease in the subject.

In a sixth aspect, disclosed herein is a computer-readable storage medium storing computer-executable program code, the program code comprising: program code for storing a dataset associated with a sample obtained from a male subject, wherein the dataset comprises data for at least one marker selected from Table 2, or a storage memory for storing a dataset associated with a sample obtained from a female subject, wherein the dataset comprises data for at least one marker selected from Table 4; and program code for analyzing the dataset to determine the abundance of the markers, wherein the levels of the markers are positively correlated or negatively correlated with a diagnosis of Anderson-Fabry Disease in the subject.

In a seventh aspect, disclosed herein is a kit for use in diagnosing Anderson-Fabry Disease (AFD) in a subject, comprising: a set of reagents comprising a plurality of reagents for determining from a sample obtained from the subject data for at least one marker selected from Table 2 or 4; and instructions for using the plurality of reagents to determine data from the samples. In some embodiments, the data is expression level data from the samples. In some embodiments, the data is protein abundance data.

In various embodiments of the above, the analyzing step further comprises applying an interpretation function to the dataset for said markers to generate a score, wherein said score is indicative of the subject's Anderson-Fabry Disease (AFD) status.

In one embodiment, the interpretation function, if the subject is male, is: score=1.62+1.56×A+0.50×B−0.15×C−0.26×D−0.36×E−0.49×F−0.67×G−1.31×H, where A is Alpha 1 antichymotrypsin; B is Isoform 1 of Sex hormone-binding globulin; C is Hemoglobin alpha-2; D is 22 kDa protein; E is Peroxiredoxin 2; F is Apolipoprotein E; G is Afamin; and H is Beta Ala His dipeptidase, and where the score cut-off is 0.54.

In another embodiment, the interpretation function, if the subject is female, is:

$$\begin{matrix}score = 1 - \frac{1}{1 + {e}^{- 2.05 \times ( \begin{matrix}- 0.49 + 0.72 \times a + 0.30 \times b + 0.25 \times c + 0.14 \times d + \\ 0.13 \times e + 0.11 \times f - 0.03 \times g - 0.24 \times h - 0.6 \times i\end{matrix} ) + 0.142}}\end{matrix} ,$$

where a is Apolipoprotein E; b is Isoform 1 of Gelsolin; c is Kallistatin; d is Peroxiredoxin 2; e is Hemoglobin alpha-2; f is Paraoxonase PON 1; g is Protein Z-dependent protease inhibitor; h is Pigment epithelium-derived factor; and I is Actin, alpha cardiac muscle 1, and where the score cut-off is 0.51.

### BRIEF DESCRIPTION OF THE DRAWINGS

FIG. 1. Biomarker discovery and replication study design.

FIGS. 2A-2D. Performance of the AFD biomarkers in the discovery and replication cohorts. FIG. 2A. Red dots indicate the biomarker score, based on the 8-protein biomarker panel, of all discovery Anderson-Fabry disease (AFD) patients on the left and all replication AFD patients on the right. The dark blue dots show the biomarker score of the healthy control (HC) individuals. The average biomarker score is shown with red and dark blue line for the AFD and HC subjects, respectively. The dotted line corresponds to the biomarker score cut-off of 0.54 for differentiating between AFD and HC subjects. FIG. 2B. The black line shows the receiver operating characteristics (ROC) curve for the discovery subjects while the green lines corresponds to the replication subjects' ROC curve. AUC stands for area under the ROC curve. FIG. 2C. The biomarker score is shown for the male subjects only and it illustrates how well the AFD and HC subjects separate in the discovery and replication cohorts. FIG. 2D. The ROC curve for the male subjects with the black and green lines corresponding to the discovery and replication ROC curves, respectively.

FIGS. 3A-3B. Performance of the female-specific AFD biomarkers in the discovery and replication cohorts. FIG. 3A. Red dots indicate the biomarker score, based on the 9-protein female-specific biomarker panel for the discovery Anderson-Fabry disease (AFD) patients who have not received enzyme replacement therapy, on the left, and female replication AFD patients on the right. The dark blue dots show the biomarker score of the healthy control (HC) individuals. The average biomarker score is shown with red and dark blue line for the AFD and HC female subjects, respectively. The dotted line corresponds to the biomarker score cut-off of 0.51 for differentiating between FD and HC subjects. FIG. 3B. The black line shows the receiver operating characteristics (ROC) curve for the discovery subjects while the green lines corresponds to the replication subjects' ROC curve. AUC stands for area under the ROC curve.

### DETAILED DESCRIPTION

Anderson-Fabry disease (AFD) is an important X-linked metabolic disease resulting in progressive central nervous system, renal and cardiac diseases with a gender-dependent phenotype. Recent epidemiologic screening for AFD suggests a prevalence of 1:3000.

As disclosed in greater detail herein, we disclose a mass spectrometry-based proteomic screen for novel plasma biomarkers in a cohort of AFD patients in comparison to matched healthy controls, and a subsequent replication study in a separate cohort of AFD patients. We further identify gender-specific biomarkers panels, which may lead to improvements in diagnosing challenging cases, such as most AFD-affected females, and variant or late-onset phenotype males.

Specifically, we used an unbiased screening proteomic approach to discover novel plasma biomarker signatures in adult patients with AFD. In discovery and validation cohorts, we used a mass spectrometry iTRAQ proteomic approach followed by multiple reaction monitoring (MRM) assays, to identify biomarkers. Of the 38 protein groups discovered by iTRAQ, 18 already had existing MRM assays, and we identified an eight-candidate biomarker panel (a 22 kDa protein, afamin, alpha 1 antichyotrypsin, apolipoprotein E, β-Ala His dipeptidase, hemoglobin α-2, isoform 1 of sex hormone-binding globulin and peroxiredoxin 2) which was very specific and sensitive for male AFD patients. In female AFD patients, we identified a nine-marker panel of proteins with only 3 proteins, apolipoprotein E, hemoglobin α-2 and peroxiredoxin 2, common to both genders, suggesting a gender-specific alteration in plasma biomarkers in patients with AFD.

Thus, disclosed herein are gender-specific plasma protein biomarker panels that are specific and sensitive for the AFD phenotype. The gender-specific panels offer important insight into potential differences in pathophysiology and prognosis between males and females.

These and other features of the present teachings will become more apparent from the description herein. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Most of the words used in this specification have the meaning that would be attributed to those words by one skilled in the art. Words specifically defined in the specification have the meaning provided in the context of the present teachings as a whole, and as are typically understood by those skilled in the art. In the event that a conflict arises between an art-understood definition of a word or phrase and a definition of the word or phrase as specifically taught in this specification, the specification shall control.

It must be noted that, as used in the specification and the appended claims, the singular forms “a,” “an,” and “the” include plural referents unless the context clearly dictates otherwise.

Terms used in the claims and specification are defined as set forth below unless otherwise specified.

The term “status” of Anderson-Fabry disease (AFD) or “AFD status” as used herein refers to the status or extent of AFD in a subject. In some contexts, AFD status may be referred to as “significant”, “non-significant”, or “possible” AFD.

“Marker” or “markers” or “biomarker,” “biomarkers,” refers generally to a molecule (typically protein, carbohydrate, lipid, or nucleic acid) that is expressed in cell or tissue, which is useful for the diagnosis of AFD. A marker in the context of the present teachings encompasses, for example, without limitation, cytokines, chemokines, growth factors, proteins, peptides, nucleic acids, oligonucleotides, and metabolites, together with their related metabolites, mutations, variants, polymorphisms, modifications, fragments, subunits, degradation products, elements, and other analytes or sample-derived measures. In the case of a nucleic acid, a marker can include any allele, including wild-types alleles, SNPs, microsatellites, insertions, deletions, duplications, and translocations. A marker can also include a peptide encoded by a nucleic acid. Markers can also include mutated proteins, mutated nucleic acids, variations in copy numbers and/or transcript variants. Markers also encompass non-blood borne factors and non-analyte physiological markers of health status, and/or other factors or markers not measured from samples (e.g., biological samples such as bodily fluids), such as clinical parameters and traditional factors for clinical assessments. Markers can also include any indices that are calculated and/or created mathematically. Markers can also include combinations of any one or more of the foregoing measurements, including temporal trends and differences.

To “analyze” includes measurement and/or detection of data associated with a marker (such as, e.g., presence or absence of a protein, or nucleic acid sequence, or constituent expression levels) in the sample (or, e.g., by obtaining a dataset reporting such measurements, as described below). In some aspects, an analysis can include comparing the measurement and/or detection of at least one marker in samples from a subject pre- and post-treatment or other control subject(s). The markers of the present teachings can be analyzed by any of various conventional methods known in the art.

A “subject” in the context of the present teachings is generally a mammal. The subject is generally a patient. The term “mammal” as used herein includes but is not limited to a human, non-human primate, dog, cat, mouse, rat, cow, horse, and pig. Mammals other than humans can be advantageously used as subjects that represent animal models of heart transplantion. A subject can be male or female.

A “sample” in the context of the present teachings refers to any biological sample that is isolated from a subject. A sample can include, without limitation, a single cell or multiple cells, fragments of cells, an aliquot of body fluid, whole blood, platelets, serum, plasma, red blood cells, white blood cells or leucocytes, endothelial cells, tissue biopsies, synovial fluid, lymphatic fluid, ascites fluid, and interstitial or extracellular fluid. The term “sample” also encompasses the fluid in spaces between cells, including gingival crevicular fluid, bone marrow, cerebrospinal fluid (CSF), saliva, mucous, sputum, semen, sweat, urine, or any other bodily fluids. “Blood sample” can refer to whole blood or any fraction thereof, including blood cells, red blood cells, white blood cells or leucocytes, platelets, serum and plasma. Samples can be obtained from a subject by means including but not limited to venipuncture, excretion, ejaculation, massage, biopsy, needle aspirate, lavage, scraping, surgical incision, or intervention or other means known in the art.

In particular aspects, the sample is a blood sample from the subject.

A “dataset” is a set of data (e.g., numerical values) resulting from evaluation of a sample. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Similarly, the term “obtaining a dataset associated with a sample” encompasses obtaining a set of data determined from at least one sample. Obtaining a dataset encompasses obtaining a sample, and processing the sample to experimentally determine the data, e.g., via measuring, mass spectrometry, antibody binding, ELISA, PCR, microarray, one or more primers, or one or more probes. The phrase also encompasses receiving a set of data, e.g., from a third party that has processed the sample to experimentally determine the dataset. Additionally, the phrase encompasses mining data from at least one database or at least one publication or a combination of databases and publications.

“Measuring” or “measurement” in the context of the present teachings refers to determining the presence, absence, quantity, amount, or effective amount of a marker or other substance (e.g., protein or nucleic acid) in a clinical or subject-derived sample, including the presence, absence, or concentration levels of such markers or substances, and/or evaluating the values or categorization of a subject's clinical parameters.

The term “expression level data” refers to a value that represents a direct, indirect, or comparative measurement of the level of expression of a polypeptide or polynucleotide (e.g., RNA or DNA). For example, “expression data” can refer to a value that represents a direct, indirect, or comparative measurement of the protein expression level of a proteomic marker of interest. In some embodiments, this measurement is performed by measuring protein concentration or protein level as described herein.

### Markers and Clinical Factors

The quantity of one or more markers of the invention can be indicated as a value. A value can be one or more numerical values resulting from evaluation of a sample under a condition. The values can be obtained, for example, by experimentally obtaining measures from a sample by an assay performed in a laboratory, or alternatively, obtaining a dataset from a service provider such as a laboratory, or from a database or a server on which the dataset has been stored, e.g., on a storage memory.

In an embodiment, the quantity of one or more markers can be one or more numerical values associated with expression levels of one or more of the markers of Tables 2 or 4 resulting from evaluation of a sample.

In an embodiment, a marker's associated value can be included in a dataset associated with a sample obtained from a subject. A dataset can include the marker expression value of two or more, three or more, four or more, five or more, six or more, seven or more, eight or more, or nine marker(s). For example, a dataset can include the expression values for one or more of the markers of Tables 2 or 4.

In an embodiment, a clinical factor can be included within a dataset. A dataset can include one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, or thirty or more overlapping or distinct clinical factor(s). A clinical factor can be, for example, the condition of a subject in the presence of a disease or in the absence of a disease, e.g., AFD. Alternatively, or in addition, a clinical factor can be the health status of a subject. Alternatively, or in addition, a clinical factor can be age, gender, clinical characteristics, organ function, functional status, morphologic characteristics, and quality of life assessments.

In another embodiment, the invention includes obtaining a sample associated with a subject, where the sample includes one or more markers. The sample can be obtained by the subject or by a third party, e.g., a medical professional. Examples of medical professionals include physicians, emergency medical technicians, nurses, first responders, psychologists, medical physics personnel, nurse practitioners, surgeons, dentists, and any other obvious medical professional as would be known to one skilled in the art. A sample can include peripheral blood cells, isolated leukocytes, or RNA extracted from peripheral blood cells or isolated leukocytes. The sample can be obtained from any bodily fluid, for example, amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour. In an example, the sample is obtained by a blood draw, where the medical professional draws blood from a subject, such as by a syringe. The bodily fluid can then be tested to determine the value of one or more markers using an assay. The value of the one or more markers can then be evaluated by the same party that performed the assay using the methods of the invention or sent to a third party for evaluation using the methods of the invention.

In some embodiments, one or more clinical factors in a subject can be assessed. In some embodiments, assessment of one or more clinical factors or variables in a subject can be combined with a marker analysis in the subject to diagnose AFD in a subject.

### Assays

Techniques, methods, tools, algorithms, reagents and other necessary aspects of assays that may be employed to detect and/or quantify a particular marker or set of markers are varied. Of significance is not so much the particular method used to detect the marker or set of markers, but what markers to detect. As is reflected in the literature, tremendous variation is possible. Once the marker or set of markers to be detected or quantified is identified, any of several techniques may be well suited, with the provision of appropriate reagents. One of skill in the art, when provided with the set of markers to be identified, will be capable of selecting the appropriate assay (for example, an ELISA, protein or antibody microarray or similar immunologic assay, or in some examples, use of an iTRAQ, iCAT, SELDI, or MRM-MS proteomic mass spectrometric based method, or a PCR based or a microarray based assay for nucleic acid markers) for performing the methods disclosed herein.

Proteins, protein complexes, or proteomic markers may be specifically identified and/or quantified by a variety of methods known in the art and may be used alone or in combination. Immunologic- or antibody-based techniques include enzyme-linked immunosorbent assay (ELISA), radioimmunoassay (RIA), western blotting, immunofluorescence, microarrays, some chromatographic techniques (i.e. immunoaffinity chromatography), flow cytometry, immunoprecipitation and the like. Such methods are based on the specificity of an antibody or antibodies for a particular epitope or combination of epitopes associated with the protein or protein complex of interest. Non-immunologic methods include those based on physical characteristics of the protein or protein complex itself. Examples of such methods include electrophoresis, some chromatographic techniques (e.g. high performance liquid chromatography (HPLC), fast protein liquid chromatography (FPLC), affinity chromatography, ion exchange chromatography, size exclusion chromatography and the like), mass spectrometry, sequencing, protease digests, and the like. Such methods are based on the mass, charge, hydrophobicity or hydrophilicity, which is derived from the amino acid complement of the protein or protein complex, and the specific sequence of the amino acids. Exemplary methods include those described in, for example, PCT Publication WO 2004/019000, WO 2000/00208, U.S. Pat. No. 6,670,194 Immunologic and non-immunologic methods may be combined to identify or characterize a protein or protein complex. Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, or the like.

Methods of producing antibodies for use in protein or antibody arrays, or other immunology based assays are known in the art. Once the marker or markers are identified and the amino acid sequence of the protein or polypeptide is identified, either by querying of a database or by having an appropriate sequence provided (for example, a sequence listing as provide herein), one of skill in the art will be able to use such information to prepare one or more appropriate antibodies and perform the selected assay.

For preparation of monoclonal antibodies directed towards a biomarker, any technique that provides for the production of antibody molecules may be used. Such techniques include, but are not limited to, hybridomas or triomas (e.g. Kohler and Milstein 1975, Nature 256:495-497; Gustafsson et al., 1991, Hum. Antibodies Hybridomas 2:26-32), human B-cell hybridoma or EBV hybridomas e.g. (Kozbor et al., 1983, Immunology Today 4:72; Cole et al., 1985, In: Monoclonal Antibodies and Cancer Therapy, Alan R. Liss, Inc., pp. 77-96). Human, or humanized antibodies may be used and can be obtained by using human hybridomas (Cote et al., 1983, Proc. Natl. Acad. Sci. USA 80:2026-2030) or by transforming human B cells with EBV virus in vitro (Cole et al., 1985, In: Monoclonal Antibodies and Cancer Therapy, Alan R. Liss, Inc., pp. 77-96). Techniques developed for the production of “chimeric antibodies” (Morrison et al., 1984, Proc. Natl. Acad. Sci. USA 81:6851-6855; Neuberger et al., 1984, Nature 312:604-608; Takeda et al., 1985, Nature 314:452-454) by splicing a sequence encoding a mouse antibody molecule specific for a particular biomarker together with a sequence encoding a human antibody molecule of appropriate biological activity may be used; such antibodies are within the scope of this invention. Techniques described for the production of single chain antibodies (U.S. Pat. No. 4,946,778) may be adapted to produce a biomarker -specific antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity for a biomarker proteins. Non-human antibodies can be “humanized” by known methods (e.g., U.S. Pat. No. 5,225,539).

Antibody fragments that contain an idiotype of a biomarker can be generated by techniques known in the art. For example, such fragments include, but are not limited to, the F(ab′)2 fragment which can be produced by pepsin digestion of the antibody molecule; the Fab′ fragment that can be generated by reducing the disulfide bridges of the F(ab′)2 fragment; the Fab fragment that can be generated by treating the antibody molecular with papain and a reducing agent; and Fv fragments. Synthetic antibodies, e.g., antibodies produced by chemical synthesis, may also be useful in the present invention.

Standard reference works described herein and known to those skilled in the relevant art describe both immunologic and non-immunologic techniques, their suitability for particular sample types, antibodies, proteins or analyses. Standard reference works setting forth the general principles of immunology and assays employing immunologic methods known to those of skill in the art include, for example: Harlow and Lane, Antibodies: A Laboratory Manual, 2d Ed., Cold Spring Harbor Laboratory Press, Cold Spring Harbor, N.Y. (1999); Harlow and Lane, Using Antibodies: A Laboratory Manual. Cold Spring Harbor Laboratory Press, New York; Coligan et al. eds. Current Protocols in Immunology, John Wiley & Sons, New York, N.Y. (1992-2006); and Roitt et al., Immunology, 3d Ed., Mosby-Year Book Europe Limited, London (1993). Standard reference works setting forth the general principles of peptide synthesis technology and methods known to those of skill in the art include, for example: Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwood et al., Oxford University Press, Oxford, United Kingdom, 2000; Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994).

A variety of methods for protein identification and quantitation are currently available, such as glycopeptide capture (Zhang et al., 2005. Mol Cell Proteomics 4:144-155), multidimensional protein identification technology (Mud-PIT) Washburn et al., 2001 Nature Biotechnology (19:242-247), and surface-enhanced laser desorption ionization (SELDI-TOF) (Hutches et al., 1993. Rapid Commun Mass Spec 7:576-580). In addition, several isotope labelling methods which allow quantification of multiple protein samples, such as isobaric tags for relative and absolute protein quantification (iTRAQ) (Ross et al., 2004 Mol Cell Proteomics 3:1154-1169); isotope coded affinity tags (ICAT) (Gygi et al., 1999 Nature Biotechnology 17:994-999), isotope coded protein labelling (ICPL) (Schmidt et al., 2004. Proteomics 5:4-15), and N-terminal isotope tagging (NIT) (Fedjaev et al., 2007 Rapid Commun Mass Spectrom 21:2671-2679; Nam et al., 2005. J Chromatogr B Analyt Technol Biomed Life Sci. 826:91-107), provide a format suitable for high-throughput performance, a trait particularly useful in biomarker screening/identification studies.

A multiplexed iTRAQ methodology was employed for identification of plasma proteomic markers. iTRAQ was first described by Ross et al., 2004 (Mol Cell Proteomics 3:1154-1169). While iTRAQ was one exemplary method used to detect the peptides, other methods described herein, for example immunological based methods such as ELISA may also be useful. Alternately, specific antibodies may be raised against the one or more proteins, isoforms, precursors, polypeptides, peptides, or portions or fragments thereof, and the specific antibody used to detect the presence of the one or more proteomic marker in the sample. Methods of selecting suitable peptides, immunizing animals (e.g. mice, rabbits or the like) for the production of antisera and/or production and screening of hybridomas for production of monoclonal antibodies are known in the art, and described in the references disclosed herein.

Another method used in the practice of the invention is MRM-MS (multiple reaction-monitoring mass spectrometry). MRM-MS based assays are known in the art and have been reviewed (Carr and Anderson, Clinical Chemistry, 54:11 (2008)).

### Interpretation Functions

In an embodiment, an interpretation function can be a function produced by a classification model. An interpretation function can also be produced by a plurality of classification models.

In an embodiment, an interpretation function derived from an elastic net model can take the form of (for males): score=1.62+1.56×A+0.50×B−0.15×C−0.26×D−0.36×E−0.49×F−0.67×G−1.31×H, where the variables and weights are as indicated in the table below, and the score cut-off is 0.54.

### AFD Biomarkers

| Protein ID | Biomarker Protein Name | Weight |
|---|---|---|
| A | Alpha 1 antichymotrypsin | 1.56 |
| B | Isoform 1 of Sex hormone-binding globulin | 0.50 |
| C | Hemoglobin alpha-2 | −0.15 |
| D | 22 kDa protein | −0.26 |
| E | Peroxiredoxin 2 | −0.36 |
| F | Apolipoprotein E | −0.49 |
| G | Afamin | −0.67 |
| H | Beta Ala His dipeptidase | −1.31 |

In an embodiment, an interpretation function derived from a support vector machine can take the form of (for females):

$$score = 1 - \frac{1}{1 + {e}^{- 2.05 \times ( \begin{matrix}- 0.49 + 0.72 \times a + 0.30 \times b + 0.25 \times c + 0.14 \times d + \\ 0.13 \times e + 0.11 \times f - 0.03 \times g - 0.24 \times h - 0.6 \times i\end{matrix} ) + 0.142}} ,$$

where the variables and weights are as indicated in the table below, and the score cut-off is 0.51.

### Female Specific Panel

| Protein ID | Biomarker Protein Name | Weight |
|---|---|---|
| a | Apolipoprotein E | 0.72 |
| b | Isoform 1 of Gelsolin | 0.30 |
| c | Kallistatin | 0.25 |
| d | Peroxiredoxin 2 | 0.14 |
| e | Hemoglobin alpha-2 | 0.13 |
| f | Paraoxonase PON 1 | 0.11 |
| g | Protein Z-dependent protease inhibitor | −0.03 |
| h | Pigment epithelium-derived factor | −0.24 |
| i | Actin, alpha cardiac muscle 1 | −0.60 |

In an embodiment, a predictive model can include a partial least squares model, an elastic net model, a logistic regression model, a linear regression model, a linear discriminant analysis model, a ridge regression model, and a tree-based recursive partitioning model. In an embodiment, a predictive model can also include Support Vector Machines, quadratic discriminant analysis, or a LASSO regression model. See Elements of Statistical Learning, Springer 2003, Hastie, Tibshirani, Friedman; which is herein incorporated by reference in its entirety for all purposes. Classification model performance can be characterized by an area under the curve (AUC). In an embodiment, classification model performance is characterized by an AUC ranging from 0.68 to 0.70. In an embodiment, classification model performance is characterized by an AUC ranging from 0.70 to 0.79. In an embodiment, classification model performance is characterized by an AUC ranging from 0.80 to 0.89. In an embodiment, classification model performance is characterized by an AUC ranging from 0.90 to 0.99. In an embodiment, classification model performance is characterized by an AUC of 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, and 1.0. Interpretation functions can be developed using combinations of informative markers as shown in the Examples below, or using a single gene whose expression is highly correlated with Anderson-Fabry Disease. In certain embodiments, methods for classifying based on a single protein are developed using elastic net or support vector machine.

In one embodiment, an interpretation function can be built by applying the formulas listed above that aggregates the combined contribution of the selected proteins and produces a single number, called the score. The score will be compared to the cut-off in order to determine if the patient has Anderson-Fabry Disease.

### Informative Marker Groups

In addition to the specific, exemplary markers identified in this application by name, accession number, or sequence, included within the scope of the invention are all operable variant sequences having at least 90% or at least 95% or at least 97% or greater identity to the exemplified sequences. The percentage of sequence identity may be determined using algorithms well known to those of ordinary skill in the art, including, e.g., BLASTn, and BLASTp, as described in Stephen F. Altschul et al., J. Mol. Biol. 215:403-410 (1990) and available at the National Center for Biotechnology Information website maintained by the National Institutes of Health. As described below, in accordance with an embodiment of the present invention, are all operable predictive models and methods for their use in scoring and optionally classifying samples that use a marker expression measurement that is now known or later discovered to be highly correlated with the expression of an exemplary marker expression value in addition to or in lieu of that exemplary marker expression value. For the purposes of the present invention, such highly correlated markers are contemplated either to be within the literal scope of the claimed inventions or alternatively encompassed as equivalents to the exemplary markers. Identification of markers having expression values that are highly correlated to those of the exemplary markers, and their use as a component of a classification model is well within the level of ordinary skill in the art.

### Computer Implementation

In one embodiment, a computer comprises at least one processor coupled to a chipset. Also coupled to the chipset are a memory, a storage device, a keyboard, a graphics adapter, a pointing device, and a network adapter. A display is coupled to the graphics adapter. In one embodiment, the functionality of the chipset is provided by a memory controller hub and an I/O controller hub. In another embodiment, the memory is coupled directly to the processor instead of the chipset.

The storage device is any device capable of holding data, like a hard drive, compact disk read-only memory (CD-ROM), DVD, or a solid-state memory device. The memory holds instructions and data used by the processor. The pointing device may be a mouse, track ball, or other type of pointing device, and is used in combination with the keyboard to input data into the computer system. The graphics adapter displays images and other information on the display. The network adapter couples the computer system to a local or wide area network.

As is known in the art, a computer can have different and/or other components than those described previously. In addition, the computer can lack certain components. Moreover, the storage device can be local and/or remote from the computer (such as embodied within a storage area network (SAN)).

As is known in the art, the computer is adapted to execute computer program modules for providing functionality described herein. As used herein, the term “module” refers to computer program logic utilized to provide the specified functionality. Thus, a module can be implemented in hardware, firmware, and/or software. In one embodiment, program modules are stored on the storage device, loaded into the memory, and executed by the processor.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

Embodiments of the entities described herein can include other and/or different modules than the ones described here. In addition, the functionality attributed to the modules can be performed by other or different modules in other embodiments. Moreover, this description occasionally omits the term “module” for purposes of clarity and convenience.

### Kits

The invention provides kits for determining quantitative expression data for one or more markers selected from Tables 2 or 4 and instructions for using the data to determine a subject's AFD status. Optionally the kit may include packaging. The kit may be used alone for diagnosing a subject's AFD status, or it may be used in conjunction with other methods for determining clinical variables, or other assays that may be deemed appropriate.

For example, the kit may comprise reagents for specific and quantitative detection of one or more than one proteomic markers selected from the markers found in Tables 2 or 4, along with instructions for the use of such reagents and methods for analyzing the resulting data. For example, the kit may comprise antibodies or fragments thereof, specific for the proteomic markers (primary antibodies), along with one or more secondary antibodies that may incorporate a detectable label; such antibodies may be used in an assay such as an ELISA. Alternately, the antibodies or fragments thereof may be fixed to a solid surface, e.g. an antibody array. The kit may be used alone for diagnosing a subject's AFD status, or it may be used in conjunction with other methods for determining clinical variables, or other assays that may be deemed appropriate. Instructions or other information useful to combine the kit results with those of other assays to provide a diagnosis of a subject's AFD status may also be provided.

### EXAMPLES

Below are examples of specific embodiments of the invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of embodiments of the invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

The goal of our work discussed below is to identify biomarkers useful for determining AFD in a subject.

### Example 1

### General Materials and Methods and Study Cohorts

### Patient Cohorts

Discovery Cohort

All patients included in the study were enrolled from Metabolic Clinics in Edmonton and Calgary, Canada. Ethics approvals were obtained from the ethics board at the University of Alberta and University of Calgary.^{13, 14} Patients with AFD and healthy control (HC) individuals were approached by the study clinical coordinators, and those who gave informed consent were enrolled in the study. A total of 32 AFD and 14 HC patients were enrolled between 2010 and 2013 to make up the discovery cohort, which is described in Table 1. Coronary artery disease (CAD) was defined as a history of MI/classic unstable angina, or pathological Q-waves (on ECG) or coronary angiogram showing >50% stenosis in any major epicardial coronaries. Cerebrovascular disease (CVD) was defined as a history of TIA/Stroke and/or brain MRI compatible with stroke/TIA or white matter changes consistent with AFD. Technical replication and recalibration was performed using the same patients and samples used for discovery but analyzed with a more clinically relevant platform, multiple reaction monitoring (MRM) mass spectrometry.

Replication Cohort

Replication was performed in AFD patients enrolled as part of the Canadian Fabry Disease Initiative (CFDI) in Halifax, Canada and HC subjects enrolled in Vancouver, Canada. Both studies were approved by Dalhousie University and the UBC Providence Health Care Research Ethics Board, respectively. The AFD and HC subjects were matched in sex, age, and other characteristics to the discovery cohort subjects, as shown in Table 1.

### Sample Collection and Processing

Blood samples from the discovery cohort were collected in BD™ P100 tubes (BD, Franklin Lakes, N.J.). The replication cohort blood samples were collected in EDTA tubes (BD, Franklin Lake, N.J.) and stored on ice until processing. For both cohorts, blood was spun down within 1 hr of collection and plasma was stored at −80° C. until selected for proteomic analysis.

### Discovery Proteomics Platform

An untargeted proteomic analysis with 8-plex isobaric tags for relative and absolute quantification (iTRAQ) was performed to identify biomarker of AFD. Analysis was performed in five phases: plasma depletion, trypsin digestion and iTRAQ labeling, high pH reversed phase fractionation, liquid chromatography (LC)-mass spectrometry (MS), and MS data analysis. The 14 most abundant plasma proteins were depleted using a custom-made 5 mL avian immunoaffinity column (Genway Biotech, San Diego, Calif., USA). Samples were digested with sequencing grade modified trypsin (Promega, Madison, Wis., USA) and labeled with iTRAQ reagents 113, 114, 115, 116, 117, 118, 119, and 121 according to the manufacturer's protocol (Applied Biosystems, Foster City, Calif., USA). Each iTRAQ set consisted of seven patient samples and one reference. The reference was randomly assigned to one of the iTRAQ labels. The study samples were randomized to the remaining seven iTRAQ labels by balancing groups between the six iTRAQ sets. High pH reversed phase fractionation was performed with an Agilent 1260 (Agilent, CA, USA) equipped with an XBridge C18 BEH300 (Waters, Mass., USA) 250 mm×4.6 mm, Sum, 300A HPLC column. The peptide solution was separated by on-line reversed phase liquid chromatography using a Thermo Scientific EASY-nanoLC II system with a reversed-phase pre-column Magic C-18AQ (Michrom BioResources Inc, Auburn, Calif.) and an in-house prepared reversed-phase nano-analytical column packed with Magic C-18AQ (Michrom BioResources Inc, Auburn, Calif.), at a flow rate of 300 nl/min. The chromatography system was coupled on-line to an LTQ Orbitrap Velos mass spectrometer equipped with a Nanospray Flex source (Thermo Fisher Scientific, Bremen, Germany). All data was analyzed using Proteome Discoverer 1.3.0.339 (Thermo Scientific, part of Thermo Fisher Scientific, Bremen, Germany) and MASCOT v2.3 (Matrix Science, Boston, Mass.) software and were searched against the Uniprot, version 20121009, human database.

### Replication Proteomics Platform

The discovery and replication cohorts' plasma samples were analyzed using Multiple Reaction Monitoring (MRM) mass spectrometry. For this study, candidate biomarker proteins, identified by iTRAQ in the discovery samples, with already existing MRM assays were measured by MRM. Additional peptides with existing MRM assay were also quantitated in the discovery and replication patient samples.

### Statistical Analysis

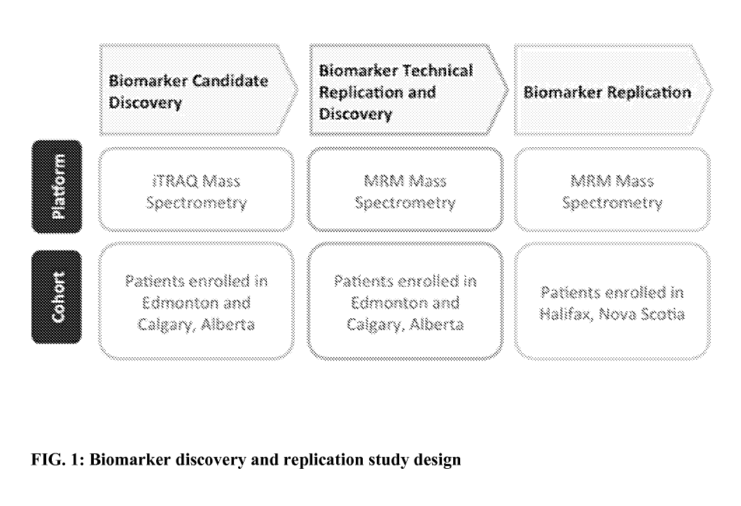

The statistical analysis of the data was performed using R (www.r-project.org) and Bioconductor (www.bioconductor.org) as per our previously published procedures.¹⁵ Briefly, the FD biomarker discovery was performed in iTRAQ, technical replication and recalibration was performed in the discovery patients in MRM, and replication was done in an external patient cohort in MRM (FIG. 1). Protein groups detected by iTRAQ in less than 75% of the discovery cohort samples were eliminated and the data were log 2 transformed. The missing values were replaced with the k nearest neighbour algorithm. The quality of the MRM data was also evaluated and those peptides with median relative ratio <0.005, median response <100, and more than two standard of deviation being out of the 80-120 range were eliminated from further analyses. As in iTRAQ, peptides present in less than 75% of the patients were eliminated from analysis. At the next step, the levels of the peptides not detected in a sample were replaced with half of the minimum peptide level detected in the rest of the patients. Following this, the MRM data was log 2 transformed and standardized. For proteins with multiple peptides measured by MRM, the level of the protein was calculated based on the peptide with highest relative ratio in the majority of the samples analyzed.

### Example 2

### Clinical Characteristics of Patients with Anderson-Fabry Disease

The discovery cohort consisted of 32 patients with AFD recruited from Edmonton and Calgary metabolic clinics, while our replication cohort was obtained from the metabolic clinic in Halifax, Canada (Table 1). Notably, the baseline characteristics and medical therapy were similar in both cohorts (Table 1). For the healthy control groups, subjects with no history of cardiovascular disease or risk-factors were selected to provide an age range and gender distribution similar to the AFD groups.

TABLE 1
Patient characteristics in the discovery and replication cohorts.

| | Discovery Cohort | | Replication Cohort | |
|---|---|---|---|---|
| | | Healthy | | Healthy |
| | AFD | Control | AFD | Control |
| N | 32 | 14 | 32 | 16 |
| Age (yr) | 42 ± 13 | 40.9 ± 13 | 42.9 ± 11.8 | 42.6 ± 12.3 |
| Gender (% Male) | 50% | 57% | 50% | 50% |
| eGFR | 96.3 ± 10.1 | — | 83.7 ± 32.2 | — |
| (mL/min/1.73 m²) | | | | |
| LVH | 50% | — | 53% | — |
| ERT | 59% | — | 63% | — |
| CAD | 0% | — | 3% | — |
| Diabetes Mellitus | 0% | — | 0% | — |
| CVD | 13% | — | 6% | — |
| ASA | 81% | — | 72% | — |
| Statin | 84% | — | 47% | — |
| ARB/ACE | 97% | — | 59% | — |
| Inhibitor | | | | |

Values represent mean±SD; eGFR=estimated GFR using the MDRD equation; LVH=left ventricular hypertrophy; ERT=enzyme replacement therapy; CAD=coronary artery disease; CVD=cerebrovascular disease; ASA=acetyl salicylic acid; ARB=AT1R blocker.

### Example 3

### iTRAQ Proteomic Fabry Disease Biomarker Discovery

AFD samples were compared with HC by means of a moderated robust t-test¹⁶ using limma Bioconductor package, developed for the analysis of ‘omic’ type of data. The proteins groups with p-value <0.05 were considered candidate biomarkers of AFD. The area under the receiver operating characteristics (AUC) curve was estimated based on leave-one-out cross-validation.

A total of 247 protein groups were detected in at least one sample. Of these, 146 were present in at least 75% of the samples. There were 38 protein groups with p-value<0.05 based on robust limma analysis. A candidate biomarker panel built with these 38 protein groups had a 0.83 cross-validation AUC.

### Example 4

### Technical Replication and Recalibration of Proteomic AFD Biomarkers in MRM

Replication of the AFD biomarkers was performed using the discovery patients analyzed by means of MRM. Since not all biomarkers had MRM assay available, the biomarker panel was recalibrated using a subset of the proteins with MRM data that were also statistically significant in the discovery MRM data between AFD and HC samples. The purpose of the recalibration was to recalculate the weights of the proteins taking into account that the panel contains fewer proteins (only those with MRM data and p-value <0.05 in MRM).

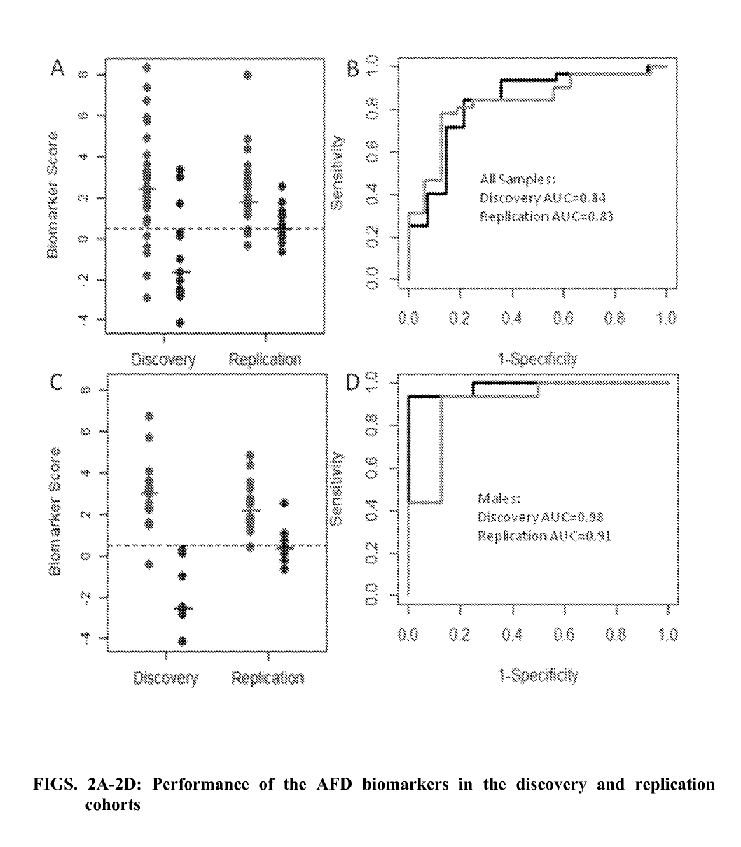

Of the 38 protein groups discovered by iTRAQ, 18 had already existing MRM assay. Of these 8 had p-value<0.05 based on robust limma analysis (Table 2). The biomarker panel was recalibrated using the 8 proteins in the MRM data such that the final model had the most separation between the AFD patients and the HC subjects. Thus, this step entailed applying elastic net classification, like in iTRAQ discovery, on the 8 proteins. The cross-validation AUC of the 8-protein final biomarker panel was 0.84, as shown on FIGS. 2A-2D. As indicated in Table 3, the biomarker panel worked almost perfectly in male patients, AUC=0.98, and had the lowest performance in females, AUC=0.65. Thus, discovery analysis was performed to identify a biomarker panel for female AFD patients.

TABLE 2
The AFD biomarker panel proteins

| | iTRAQ | | MRM | | |
|---|---|---|---|---|---|
| | | Fold | | Fold | Direction (FD |
| Protein | P-value | Change | P-value | Change | relative to HC) |
| 22 kDa protein | 0.02 | 1.32 | 0.01 | 1.45 | down |
| Afamin | 0.00 | 1.59 | 0.01 | 1.23 | down |
| Alpha 1 | 0.04 | 1.11 | 0.02 | 1.23 | up |
| antichymotrypsin | | | | | |
| Apolipoprotein E | 0.00 | 1.61 | 0.00 | 1.42 | down |
| Beta Ala | 0.01 | 1.19 | 0.01 | 1.43 | down |
| His dipeptidase | | | | | |
| Hemoglobin | 0.03 | 1.61 | 0.02 | 1.79 | down |
| alpha-2 | | | | | |
| Isoform 1 | 0.03 | 1.13 | 0.04 | 1.60 | up |
| of Sex hormone- | | | | | |
| binding globulin | | | | | |
| Peroxiredoxin 2 | 0.00 | 1.56 | 0.00 | 1.55 | down |

TABLE 3
Performance characteristics of the AFD biomarker panel for all
samples and for males and females separately.

| | Performance | | | |
|---|---|---|---|---|
| Cohort | Characteristic | All Samples | Females | Males |
| Discovery | AUC | 0.84 | 0.65 | 0.98 |
| | Sensitivity | 84% | 75% | 94% |
| | Specificity | 79% | 50% | 100% |
| Replication | AUC | 0.83 | 0.76 | 0.91 |
| | Sensitivity | 84% | 75% | 94% |
| | Specificity | 63% | 63% | 63% |

### Example 5

### MRM Proteomic Female-Specific AFD Biomarker Discovery

Since the current diagnostic methods of AFD are not working very well for female patients, a separate discovery analysis was performed on the MRM data by focusing on the comparison of female FD patients who are not on enzyme replacement therapy (ERT) and female HCs. This analysis was similar to the biomarker discovery described for iTRAQ but it was performed in the MRM data of the discovery cohort.

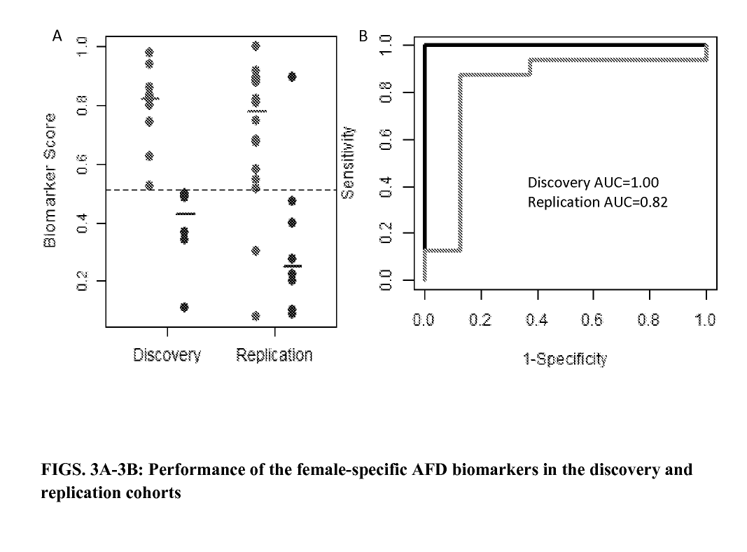

A biomarker discovery was performed using the MRM data specifically on female AFD patients, which is the hardest group to diagnose using the current clinically available tests. A total of 306 peptides corresponding to 125 proteins were measured by MRM. Of these, 137 peptides (71 proteins) passed quality control. A total of 70 proteins were present in 75% of the samples, which were analyzed with robust limma moderated t-test. The best biomarker panel consisted of 9 proteins, as listed in Table 4, and was built with support vector machine (SVM) classification method. The cross-validation AUC of this panel was 1.00 (FIGS. 3A-3B; Table 5).

TABLE 4
The female-specific AFD
biomarker panel proteins.

| | | | | Direction |
|---|---|---|---|---|
| | Peptide | | | (AFD |
| | (SEQ ID | | Fold | relative |
| Protein | NOS: 1-9) | P-value | Change | to HC) |
| Actin, alpha | SYELPDGQV | 0.03 | 1.43 | Up |
| cardiac muscle 1 | ITIGNER | | | |
| Apolipo- | AATVGSLAG | 0.04 | 1.27 | Down |
| protein E | QPLQER | | | |
| Hemoglobin | TYFPHFDLS | 0.03 | 1.70 | Up |
| alpha-2 | HGSAQVK | | | |
| Isoform 1 | EVQGFESAT | 0.01 | 1.35 | Down |
| of Gelsolin | FLGYFK | | | |
| Kallistatin | LGFTDLFSK | 0.03 | 1.32 | Down |
| Paraoxonase | SFNPNSPGK | 0.05 | 1.51 | Down |
| PON 1 | | | | |
| Peroxiredoxin 2 | GLFIIDGK | 0.07 | 1.76 | Down |
| Pigment | TVQAVLTVP | 0.04 | 1.25 | Up |
| epithelium- | K | | | |
| derived factor | | | | |
| Protein | ETSNFGFSL | 0.06 | 1.21 | Up |
| Z-dependent | LR | | | |
| protease | | | | |
| inhibitor | | | | |

TABLE 5
Performance characteristics of the female-specific AFD biomarker
panel.

| | | Replication |
|---|---|---|
| | Discovery Females | Females |
| AUC | 1.00 | 0.82 |
| Sensitivity | 100% | 88% |
| Specificity | 100% | 88% |

### Example 6

### Replication of AFD Biomarkers in a Separate Cohort

The final AFD biomarker panel built in MRM was tested in the 48 subject recalibration and replication cohort (32 AFD and 16 HC). The female-specific AFD biomarker panel was also replicated in the female patients from the replication cohort (16 AFD and 8 HC).

We used a replication cohort of patients with AFD from Halifax, Nova Scotia. The test AUC of the 8-protein final biomarker panel was 0.83, as shown on FIGS. 2A-2D. As indicated in Table 3, the biomarker panel still worked very well in male patients, test AUC=0.91, and had the lower performance in females, AUC=0.76.

### Example 6

### Replication of Female-Specific AFD Biomarkers in a Separate Cohort

The 9-protein female-specific biomarker panel was tested in 16 AFD and 8 HC female subjects from the replication cohort by applying the panel and associated weights as identified in the discovery cohort. The replication AUC in this cohort of 24 subjects was 0.82 (FIGS. 3A-3B). When the cut-off set in the discovery cohort, to maximize Youden's index, was applied the sensitivity and specificity in the replication cohort were 88% and 88%, respectively (Table 5).

### Discussion

In this study, we report the discovery and subsequent replication of a novel set of plasma protein markers for AFD. AFD is an important metabolic disorder with deleterious effects on many organ systems that culminates in end-organ failure, and substantial morbidity and mortality. On a global basis, AFD is now increasingly being recognized as a small but significant contributor to cardiovascular morbidity.¹⁷⁻²⁰ In particular, variant and late-onset phenotypes with primarily cardiovascular manifestations are being recognized as an important cause of cardiomyopathies.^{21, 22} Given that early identification and treatment of AFD patients with ERT can reduce progression of heart disease and renal dysfunction, considerable research has focused on improving the existing diagnostic algorithm.^{8, 23-26} In order to generate a robust biomarker panel, we used a proteomic discovery approach in a cohort of 32 AFD patients in comparison to 14 healthy control individuals, all from Edmonton and Calgary in the province of Alberta, Canada. We then replicated these results in a cohort of 32 AFD patients from Halifax, Canada in comparison to 16 healthy individuals from Vancouver, Canada. The two AFD cohorts were closely matched to their associated control groups in terms of age and gender, and the AFD cohorts were treated and managed concordantly with a similar risk profile. The emergence of a common biomarker panel in both cohorts suggests that these biomarkers reflect the presence of AFD regardless of optimum medical therapy.

Following discovery in the Alberta AFD cohort, we replicated the results in the Halifax AFD cohort to generate an eight-peptide biomarker panel that contained markers that had achieved a significance level of at least 0.05 and could reliably be detected in both proteomic platforms used. The identified peptides have diverse biological roles, including blood transport and composition, protease activity, and antioxidant effects. All together these reflect the complex multisystem involvement that is characteristic of AFD. In males the eight-peptide biomarker panel performed very well at separating AFD from controls with an area under the receiver operating characteristics curve of 0.98 in the discovery cohort and 0.91 in the replication cohort. Our eight-peptide panel for the whole AFD group was not optimal for female patients, which is likely driven by a gender-specific metabolic response²⁷ and in the phenotypic manifestations^{28, 29} of AFD. We thus generated a nine-peptide panel specific to females, which may lead both to improved diagnostic catchment, and to better prognostication in female patients with AFD. Our female-specific panel contained more peptides with roles in protease activity and antioxidant effects, as well as cytoskeletal composition, which was a unique feature as compared to the whole AFD group. The female-specific panel separated AFD from controls with an AUC operating characteristics curve of 1.00 in the discovery cohort and 0.81 in the replication cohort, and may provide an unprecedented ability to detect AFD in female heterozygotes. Presently, female heterozygotes represent the most challenging AFD patient group, because their symptoms may range from absent to severe, but initially appear mild. There is evidence that the majority of affected females do develop clinically significant disease; however, their constellation of symptoms is frequently variable.^{10, 30, 31} Alpha-galactosidase A activity assays are not reliable in females, as the range in affected individuals ranges from very low to normal. Genetic testing is the present standard for confirming AFD in females. However, biomarker panels, such as the nine-peptide panel we have identified, will be helpful in the case of ambiguous mutations, or genetic lesions that confound genetic analysis, such as large scale deletions.^{12, 32}

Our data indicate that differences between male hemizygotes and female heterozygotes are manifested in differences in pathophysiology in AFD. The male and female panels share three proteins: apolipoprotein E (ApoE), a constituent of chylomicrons involved in cholesterol shuttling; hemoglobin alpha-2 (Hbα₂), a constituent of normal adult hemoglobin; and peroxiredoxin 2 (Prx2), an abundant thiol protein in erythrocytes that provides antioxidant effects. ApoE and Prx2 are both decreased in male and female AFD patients, which might indicate a reduction in these patients' abilities to shuttle blood lipids, and deal with oxidative stress, respectively. Interestingly, Hbα₂ is decreased in males but increased in females, which may reflect the difference in anemia prevalence between male and female AFD patients that is consistent with the lower prevalence of severe renal complications in AFD females.^{30, 33, 34} The male biomarker panel contains afamin and isoform 1 of sex hormone-binding globulin, general and sex-hormone transport proteins, respectively, as well as alpha 1 antichyotrypsin and carnosinase, a protease and protease inhibitor, respectively. The female biomarker panel, meanwhile, contains kallistatin and protein-Z dependent protease inhibitor, which are both protease inhibitors; however, cardiac-specific alpha actin and isoform 1 of gelsolin, a constituent of the cardiac cytoskeleton and an actin capping and severing protein, respectively, are also present. This suggests the integrity of the cardiac cytoskeleton is modulated in females with AFD in a more consistent manner than the males with AFD we studied.

Much of the effort to find urinary and plasma biomarkers in AFD has been metabolomic in nature and has focused largely on Gb3 and its metabolites, including globotriaosylsphingosine (lyso-Gb3).^{12, 35-44} Plasma lyso-Gb3 levels are reduced in AFD patients after initiation of ERT, while urinary lyso-Gb3 is correlated to some indices of kidney function.³⁶⁻³⁹ Recently, however, Mitobe et al. discovered a subset of patients with late-onset AFD due to the M296I mutation whose plasma lyso-Gb3 levels were not increased, which highlights the potential pitfalls of not expanding the diagnostic algorithm to include new biomarkers.⁴⁵ With regards to two important characteristics of biomarkers, correlating to indices of disease severity and offering pathophysiological insight, metabolic AFD biomarkers are insufficient. Indeed, Gb3 and its derivatives may not always reflect disease severity, particularly in variant cardiac and renal phenotypes.^{36, 39}.

Proteomic analyses, meanwhile, offer a potential complement to metabolomic analyses, which, in concert, may generate a more complete picture of the pathophysiology of AFD.^{7, 46-49} In comparing our results to proteomic analysis in peripheral blood mononuclear cells (PBMCs), similar themes emerge, whereby cell signaling molecules are altered, but there is no direct overlap.⁴⁹ Further, the AFD proteome in PBMCs implicates inflammation, whereas our data implicates oxidative stress, although, implying that these processes are dysregulated in tandem. Proteomic analysis may also reflect changes in serum proteins in response to ERT in pediatric AFD patients.⁷ Interestingly, when taking our data together with published reports of urinary proteomic changes in AFD, there are changes in mediators of protease activity, cell signaling molecules, and blood composition and lipid shuttling molecules, but urinary proteomes also implicate ECM remodeling through peptide fragments of collagens, while our data implicate cytoskeletal changes, at least in females.^{47, 48}

### REFERENCES

1. Clarke J T. Narrative review: Fabry disease. Ann Intern Med. 2007; 146:425-433.
2. Spada M, Pagliardini S, Yasuda M, Tukel T, Thiagarajan G, Sakuraba H, et al. High incidence of later-onset fabry disease revealed by newborn screening. Am J Hum Genet. 2006; 79:31-40.
3. Lin H Y, Chong K W, Hsu J H, Yu H C, Shih C C, Huang C H, et al. High incidence of the cardiac variant of fabry disease revealed by newborn screening in the taiwan chinese population. Circ Cardiovasc Genet. 2009; 2:450-456.
4. Mechtler T P, Stary S, Metz T F, De Jesus V R, Greber-Platzer S, Pollak A, et al. Neonatal screening for lysosomal storage disorders: Feasibility and incidence from a nationwide study in austria. Lancet. 2012; 379:335-341.
5. Inoue T, Hattori K, Ihara K, Ishii A, Nakamura K, Hirose S. Newborn screening for fabry disease in japan: Prevalence and genotypes of fabry disease in a pilot study. J Hum Genet. 2013; 58:548-552.
6. Mehta A, Clarke J T, Giugliani R, Elliott P, Linhart A, Beck M, et al. Natural course of fabry disease: Changing pattern of causes of death in fos-fabry outcome survey. J Med Genet. 2009; 46:548-552.
7. Moore D F, Krokhin O V, Beavis R C, Ries M, Robinson C, Goldin E, et al. Proteomics of specific treatment-related alterations in fabry disease: A strategy to identify biological abnormalities. Proc Natl Acad Sci USA. 2007; 104:2873-2878.
8. Cox T M. Biomarkers in lysosomal storage diseases. In: Mehta A, Beck M, Sunder-Plassmann G, eds. Fabry disease: Perspectives from 5 years of fos. Oxford; 2006.
9. Klingler D, Hardt M. Targeting proteases in cardiovascular diseases by mass spectrometry-based proteomics. Circ Cardiovasc Genet. 2012; 5:265.
10. Laney D A, Bennett R L, Clarke V, Fox A, Hopkin R J, Johnson J, et al. Fabry disease practice guidelines: Recommendations of the national society of genetic counselors. J Genet Couns. 2013; 22:555-564.
11. Havndrup O, Christiansen M, Stoevring B, Jensen M, Hoffman-Bang J, Andersen P S, et al. Fabry disease mimicking hypertrophic cardiomyopathy: Genetic screening needed for establishing the diagnosis in women. Eur J Heart Fail. 2010; 12:535-540.
12. Niemann M, Rolfs A, Stork S, Bijnens B, Breunig F, Beer M, et al. Gene mutations versus clinically relevant phenotypes: Lyso-gb3 defines fabry disease. Circ Cardiovasc Genet. 2014; 7:8-16.
13. Thompson R B, Chow K, Khan A, Chan A, Shanks M, Paterson I, et al. T1 mapping with cardiovascular mri is highly sensitive for fabry disease independent of hypertrophy and sex. Circ Cardiovasc Imaging. 2013; 6:637-645.
14. Shanks M, Thompson R B, Paterson I D, Putko B, Khan A, Chan A, et al. Systolic and diastolic function assessment in fabry disease patients using speckle-tracking imaging and comparison with conventional echocardiographic measurements. J Am Soc Echocardiogr. 2013; 26:1407-1414.
15. Cohen Freue G V, Meredith A, Smith D, Bergman A, Sasaki M, Lam K K, et al. Computational biomarker pipeline from discovery to clinical implementation: Plasma proteomic biomarkers for cardiac transplantation. PLoS Comput Biol. 2013; 9:e1002963.
16. Smyth G K. Linear models and empirical bayes methods for assessing differential expression in microarray experiments. Stat Appl Genet Mol Biol. 2004; 3:Article 3.
17. Nakao S, Takenaka T, Maeda M, Kodama C, Tanaka A, Tahara M, et al. An atypical variant of fabry's disease in men with left ventricular hypertrophy. N Engl J Med. 1995; 333:288-293.
18. Sachdev B, Takenaka T, Teraguchi H, Tei C, Lee P, McKenna W J, et al. Prevalence of anderson-fabry disease in male patients with late onset hypertrophic cardiomyopathy. Circulation. 2002; 105:1407-1411.
19. Chimenti C, Pieroni M, Morgante E, Antuzzi D, Russo A, Russo M A, et al. Prevalence of fabry disease in female patients with late-onset hypertrophic cardiomyopathy. Circulation. 2004; 110:1047-1053.
20. Elliott P, Baker R, Pasquale F, Quarta G, Ebrahim H, Mehta A B, et al. Prevalence of anderson-fabry disease in patients with hypertrophic cardiomyopathy: The european anderson-fabry disease survey. Heart. 2011; 97:1957-1960.
21. Arbustini E, Narula N, Dec G W, Reddy K S, Greenberg B, Kushwaha S, et al. The moge(s) classification for a phenotype-genotype nomenclature of cardiomyopathy: Endorsed by the world heart federation. J Am Coll Cardiol. 2013; 62:2046-2072.
22. Rao D A, Lakdawala N K, Miller A L, Loscalzo J. Clinical problem-solving. In the thick of it. N Engl J Med. 2013; 368:1732-1738.
23. Weidemann F, Niemann M, Breunig F, Herrmann S, Beer M, Stork S, et al. Long-term effects of enzyme replacement therapy on fabry cardiomyopathy: Evidence for a better outcome with early treatment. Circulation. 2009; 119:524-529.
24. Cuccurullo M, Beneduci A, Anand S, Mignani R, Cianciaruso B, Bachi A, et al. Fabry disease: Perspectives of urinary proteomics. J Nephrol. 2010; 23 Suppl 16:S199-212.
25. Aerts J M, Kallemeijn W W, Wegdam W, Joao Ferraz M, van Breemen M J, Dekker N, et al. Biomarkers in the diagnosis of lysosomal storage disorders: Proteins, lipids, and inhibodies. J Inherit Metab Dis. 2011; 34:605-619.
26. Weidemann F, Breunig F, Beer M, Sandstede J, Turschner O, Voelker W, et al. Improvement of cardiac function during enzyme replacement therapy in patients with fabry disease: A prospective strain rate imaging study. Circulation. 2003; 108:1299-1301.
27. Durant B, Forni S, Sweetman L, Brignol N, Meng X L, Benjamin E R, et al. Sex differences of urinary and kidney globotriaosylceramide and lyso-globotriaosylceramide in fabry mice. J Lipid Res. 2011; 52:1742-1746.
28. Weidemann F, Breunig F, Beer M, Sandstede J, Stork S, Voelker W, et al. The variation of morphological and functional cardiac manifestation in fabry disease: Potential implications for the time course of the disease. Eur Heart J. 2005; 26:1221-1227.
29. Niemann M, Herrmann S, Hu K, Breunig F, Strotmann J, Beer M, et al. Differences in fabry cardiomyopathy between female and male patients: Consequences for diagnostic assessment. JACC Cardiovasc Imaging. 2011; 4:592-601.
30. MacDermot K D, Holmes A, Miners A H. Anderson-fabry disease: Clinical manifestations and impact of disease in a cohort of 60 obligate carrier females. J Med Genet. 2001; 38:769-775.
31. Wang R Y, Lelis A, Mirocha J, Wilcox W R. Heterozygous fabry women are not just carriers, but have a significant burden of disease and impaired quality of life. Genet Med. 2007; 9:34-45.
32. Feldt-Rasmussen U, Dobrovolny R, Nazarenko I, Ballegaard M, Hasholt L, Rasmussen A K, et al. Diagnostic dilemma: A young woman with fabry disease symptoms, no family history, and a “sequencing cryptic” alpha-galactosidase a large deletion. Mol Genet Metab. 2011; 104:314-318.
33. MacDermot K D, Holmes A, Miners A H. Anderson-fabry disease: Clinical manifestations and impact of disease in a cohort of 98 hemizygous males. J Med Genet. 2001; 38:750-760.
34. Kleinert J, Dehout F, Schwarting A, de Lorenzo A G, Ricci R, Kampmann C, et al. Anemia is a new complication in fabry disease: Data from the fabry outcome survey. Kidney Int. 2005; 67:1955-1960.
35. Aerts J M, Groener J E, Kuiper S, Donker-Koopman W E, Strijland A, Ottenhoff R, et al. Elevated globotriaosylsphingosine is a hallmark of fabry disease. Proc Natl Acad Sci USA. 2008; 105:2812-2817.
36. Togawa T, Kodama T, Suzuki T, Sugawara K, Tsukimura T, Ohashi T, et al. Plasma globotriaosylsphingosine as a biomarker of fabry disease. Mol Genet Metab. 2010; 100:257-261.
37. Boutin M, Gagnon R, Lavoie P, Auray-Blais C. Lc-ms/ms analysis of plasma lyso-gb3 in fabry disease. Clin Chim Acta. 2012; 414:273-280.
38. van Breemen M J, Rombach S M, Dekker N, Poorthuis B J, Linthorst G E, Zwinderman A H, et al. Reduction of elevated plasma globotriaosylsphingosine in patients with classic fabry disease following enzyme replacement therapy. Biochim Biophys Acta. 2011; 1812:70-76.
39. Auray-Blais C, Ntwari A, Clarke J T, Warnock D G, Oliveira J P, Young S P, et al. How well does urinary lyso-gb3 function as a biomarker in fabry disease? Clin Chim Acta. 2010; 411:1906-1914.
40. Paschke E, Fauler G, Winkler H, Schlagenhauf A, Plecko B, Erwa W, et al. Urinary total globotriaosylceramide and isoforms to identify women with fabry disease: A diagnostic test study. Am J Kidney Dis. 2011; 57:673-681.
41. Kruger R, Tholey A, Jakoby T, Vogelsberger R, Monnikes R, Rossmann H, et al. Quantification of the fabry marker lysogb3 in human plasma by tandem mass spectrometry. J Chromatogr B. 2012; 883-884:128-135.
42. Auray-Blais C, Boutin M. Novel gb(3) isoforms detected in urine of fabry disease patients: A metabolomic study. Curr Med Chem. 2012; 19:3241-3252.
43. Dupont F O, Gagnon R, Boutin M, Auray-Blais C. A metabolomic study reveals novel plasma lyso-gb3 analogs as fabry disease biomarkers. Curr Med Chem. 2013; 20:280-288.
44. Gold H, Mirzaian M, Dekker N, Joao Ferraz M, Lugtenburg J, Codee J D, et al. Quantification of globotriaosylsphingosine in plasma and urine of fabry patients by stable isotope ultraperformance liquid chromatography-tandem mass spectrometry. Clin Chem. 2013; 59:547-556.
45. Mitobe S, Togawa T, Tsukimura T, Kodama T, Tanaka T, Doi K, et al. Mutant alpha-galactosidase a with m296i does not cause elevation of the plasma globotriaosylsphingosine level. Mol Genet Metab. 2012; 107:623-626.
46. Vojtova L, Zima T, Tesar V, Michalova J, Prikryl P, Dostalova G, et al. Study of urinary proteomes in anderson-fabry disease. Ren Fail. 2010; 32:1202-1209.
47. Kistler A D, Siwy J, Breunig F, Jeevaratnam P, Scherl A, Mullen W, et al. A distinct urinary biomarker pattern characteristic of female fabry patients that mirrors response to enzyme replacement therapy. Plos One. 2011; 6:e20534.
48. Lepedda A J, Fancellu L, Zinellu E, De Muro P, Nieddu G, Deiana G A, et al. Urine bikunin as a marker of renal impairment in fabry's disease. Biomed Res Int. 2013; 2013:205948.
49. Cigna D, D'Anna C, Zizzo C, Francofonte D, Sorrentino I, Colomba P, et al. Alteration of proteomic profiles in pbmc isolated from patients with fabry disease: Preliminary findings. Mol Biosyst. 2013; 9:1162-1168.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A method for diagnosing Anderson-Fabry Disease (AFD) in a male subject, comprising:
obtaining a dataset associated with a sample obtained from the male subject, wherein the dataset comprises at least one marker selected from Table 2;
analyzing the dataset to determine data for the markers, wherein the data is positively correlated or negatively correlated with a diagnosis of Anderson-Fabry Disease in the male subject.

2. The method of claim 1, wherein the dataset comprises data for at least two, three, four, five, six, seven, or eight markers.

3. The method of claim 2, further comprising determining the diagnosis of Anderson-Fabry Disease in the subject according to the relative number of positively correlated and negatively correlated marker expression level data present in the dataset.

4. A method for diagnosing Anderson-Fabry Disease (AFD) in a female subject, comprising:
obtaining a dataset associated with a sample obtained from the female subject, wherein the dataset comprises at least one marker selected from Table 4;
analyzing the dataset to determine data for the markers, wherein the data is positively correlated or negatively correlated with a diagnosis of Anderson-Fabry Disease in the female subject.

5. The method of claim 4, wherein the dataset comprises data for at least two, three, four, five, six, seven, eight or nine markers.

6. The method of claim 4, further comprising determining the diagnosis of Anderson-Fabry Disease in the subject according to the relative number of positively correlated and negatively correlated marker expression level data present in the dataset.

7. The method of claim 1 or 4, wherein the sample obtained from the subject is a blood sample.

8. The method of claim 1 or 4, wherein the data is protein expression data.

9. The method of claim 8, wherein the protein expression data is obtained using an antibody.

10. The method of claim 9, wherein the antibody is labeled.

11. The method of claim 1 or 4, wherein the method is implemented using one or more computers.

12. The method of claim 1 or 4, wherein the dataset is obtained stored on a storage memory.

13. The method of claim 1 or 4, wherein obtaining the dataset comprises receiving the dataset directly or indirectly from a third party that has processed the sample to experimentally determine the dataset.

14. The method of claim 1 or 4, wherein the subject is a human subject.

15. The method of claim 1 or 4, further comprising assessing a clinical variable; and combining the assessment with the analysis of the dataset to diagnose Anderson-Fabry Disease (AFD) in the subject.

16. A method for predicting the likelihood of acute cardiac allograft rejection in a subject, comprising:
obtaining a sample from a male subject, wherein the sample comprises at least one marker selected from Table 2, or obtaining a sample from a female subject, wherein the sample comprises at least one marker selected from Table 4;
contacting the sample with a reagent;
generating a complex between the reagent and the markers;
detecting the complex to obtain a dataset associated with the sample, wherein the dataset comprises expression level data for the markers; and
analyzing the expression level data for the markers, wherein the expression level of the markers is positively correlated or negatively correlated with a diagnosis of Anderson-Fabry Disease in the subject.

17. A computer-implemented method for diagnosing Anderson-Fabry Disease in a subject, comprising:
storing, in a storage memory, a dataset associated with a sample obtained from a male subject, wherein the dataset comprises data for at least one marker selected from Table 2, or storing, in a storage memory, a dataset associated with a sample obtained from a female subject, wherein the dataset comprises data for at least one marker selected from Table 4; and
analyzing, by a computer processor, the dataset to determine the expression levels of the markers, wherein the expression levels are positively correlated or negatively correlated with a diagnosis of Anderson-Fabry Disease in the subject.

18. A system for diagnosing Anderson-Fabry Disease in a subject, the system comprising:
a storage memory for storing a dataset associated with a sample obtained from a male subject, wherein the dataset comprises data for at least one marker selected from Table 2, or a storage memory for storing a dataset associated with a sample obtained from a female subject, wherein the dataset comprises data for at least one marker selected from Table 4; and
a processor communicatively coupled to the storage memory for analyzing the dataset to determine the expression levels of the markers, wherein the expression levels are positively correlated or negatively correlated with a diagnosis of Anderson-Fabry Disease in the subject.

19. A computer-readable storage medium storing computer-executable program code, the program code comprising:
program code for storing a dataset associated with a sample obtained from a male subject, wherein the dataset comprises data for at least one marker selected from Table 2, or a storage memory for storing a dataset associated with a sample obtained from a female subject, wherein the dataset comprises data for at least one marker selected from Table 4; and
program code for analyzing the dataset to determine the expression levels of the markers, wherein the expression levels of the markers are positively correlated or negatively correlated with a diagnosis of Anderson-Fabry Disease in the subject.

20. A kit for use in diagnosing Anderson-Fabry Disease (AFD) in a subject, comprising:
a set of reagents comprising a plurality of reagents for determining from a sample obtained from the subject data for at least one marker selected from Table 2 or 4; and
instructions for using the plurality of reagents to determine data from the samples.

21. The kit of claim 20, wherein the data is expression level data from the samples.

22. The method of any one of claims 1, 4, 16, 17, 18, and 19, wherein said analyzing step further comprises applying an interpretation function to the dataset for said markers to generate a score, wherein said score compared to the cut-off is indicative of the subject's Anderson-Fabry Disease (AFD) status.

23. The method of claim 22, wherein said interpretation function, if the subject is male, is: score=1.62+1.56×A+0.50×B−0.15×C−0.26×D−0.36×E−0.49×F−0.67×G−1.31×H, where A is Alpha 1 antichymotrypsin; B is Isoform 1 of Sex hormone-binding globulin; C is Hemoglobin alpha-2; D is 22 kDa protein; E is Peroxiredoxin 2; F is Apolipoprotein E; G is Afamin; and H is Beta Ala His dipeptidase, and where the score cut-off is 0.54.

24. The method of claim 22, wherein said interpretation function, if the subject is female, is:
$$score = 1 - \frac{1}{1 + {e}^{- 2.05 \times ( \begin{matrix}- 0.49 + 0.72 \times a + 0.30 \times b + 0.25 \times c + 0.14 \times d + \\ 0.13 \times e + 0.11 \times f - 0.03 \times g - 0.24 \times h - 0.6 \times i\end{matrix} ) + 0.142}} ,$$
where a is Apolipoprotein E; b is Isoform 1 of Gelsolin; c is Kallistatin; d is Peroxiredoxin 2; e is Hemoglobin alpha-2; f is Paraoxonase PON 1; g is Protein Z-dependent protease inhibitor; h is Pigment epithelium-derived factor; and I is Actin, alpha cardiac muscle 1, and where the score cut-off is 0.51.


SEQUENCE LISTING (9 sequences)

SEQ ID NO: 1 - protein, 16 aa, Artificial Sequence
  synthetic biomarker peptide - actin, alpha cardiac muscle 1
SYELPDGQVITIGNER

SEQ ID NO: 2 - protein, 15 aa, Artificial Sequence
  synthetic biomarker peptide - apolipoprotein E
AATVGSLAGQPLQER

SEQ ID NO: 3 - protein, 16 aa, Artificial Sequence
  synthetic biomarker peptide - hemoglobin alpha-2
TYFPHFDLSHGSAQVK

SEQ ID NO: 4 - protein, 15 aa, Artificial Sequence
  synthetic biomarker peptide - isoform 1 of gelsolin
EVQGFESATFLGYFK

SEQ ID NO: 5 - protein, 9 aa, Artificial Sequence
  synthetic biomarker peptide - kallistatin
LGFTDLFSK

SEQ ID NO: 6 - protein, 9 aa, Artificial Sequence
  synthetic biomarker peptide - paraoxonase PON 1
SFNPNSPGK

SEQ ID NO: 7 - protein, 8 aa, Artificial Sequence
  synthetic biomarker peptide - peroiredoxin 2
GLFIIDGK

SEQ ID NO: 8 - protein, 10 aa, Artificial Sequence
  synthetic biomarker peptide - pigment epithelium-derived factor
TVQAVLTVPK

SEQ ID NO: 9 - protein, 11 aa, Artificial Sequence
  synthetic biomarker peptide - protein Z-dependent protease inhibitor
ETSNFGFSLLR